(19) United States
(12) Patent Application Publication
(10) Pub. No.: US 2023/0238148 A1
(43) Pub. Date: Jul. 27, 2023

(54) INFORMATION PROCESSING DEVICE, INFORMATION PROCESSING METHOD, AND COMPUTER PROGRAM

(71) Applicant: TERUMO KABUSHIKI KAISHA, Tokyo (JP)

(72) Inventor: Takanori TOMINAGA, Hadano-shi (JP)

(73) Assignee: TERUMO KABUSHIKI KAISHA, Tokyo (JP)

(21) Appl. No.: 18/192,311

(22) Filed: Mar. 29, 2023

Related U.S. Application Data
(63) Continuation of application No. PCT/JP2021/035310, filed on Sep. 27, 2021.

(30) Foreign Application Priority Data
Sep. 29, 2020 (JP) 2020-163919

(51) Int. Cl. G16H 50/70; G16H 10/60

(52) U.S. Cl. CPC G16H 50/70; G16H 10/60

(57) ABSTRACT

An information processing device, information processing method, and program which, acquire medical information collected when catheter treatment is performed on a patient; calculate a score related to a prognosis for each of a plurality of complications that may occur according to the catheter treatment on the basis of the acquired medical information; identify one or a plurality of complications at risk among the plurality of complications on the basis of the calculated score; retrieve a similar case having a score similar to the score calculated from a storage unit configured to store information regarding a complication that has occurred in the past and a score related to the prognosis calculated for the complication in association with each other; and output information on the complication identified by the identification unit and information on the similar case retrieved from the storage unit.

### CROSS-REFERENCES TO RELATED APPLICATIONS

This application is a continuation of International Application PCT/JP2021/035310 filed on Sep. 27, 2021, which claims priority to Japanese Application No. 2020-163919 filed on Sep. 29, 2020, the entire content of both of which is incorporated herein by reference.

### TECHNOLOGICAL FIELD

The present disclosure generally relates to an information processing device, an information processing method, and a computer program.

### BACKGROUND DISCUSSION

In the medical field, complications caused by medical practice such as surgery and examination become a problem. Accordingly, various systems for assisting prevention of complications have been proposed.

For example, Japanese Patent Application Publication No. 2014-200549 A discloses a medical image processing device or the like that detects a region of a peripheral tissue existing around an aortic valve of a heart from a medical image captured by an X-ray computed tomography (CT) device, arranges a model image of a prosthetic valve to be replaced with the aortic valve in the medical image, and evaluates a risk of a complication from a distance between the region of the peripheral tissue and the model image of the prosthetic valve.

According to Japanese Patent Application Publication No. 2014-200549 A, the risk of complications is evaluated by simple pattern matching based on the distance in the image, and it cannot be said that the accuracy is necessarily good.

### SUMMARY

An information processing device, an information processing method, and a computer program capable of evaluating a risk regarding complications on the basis of various medical information collected at the time of catheter treatment.

An information processing device according to one aspect includes: a processor configured to: acquire medical information collected when catheter treatment is performed on a patient; calculate a score related to a prognosis for each of a plurality of complications that may occur according to the catheter treatment on the basis of the acquired medical information; identify one or more complications at risk among the plurality of complications on the basis of the calculated score; retrieve a similar case having a score similar to the score calculated from a storage unit configured to store information regarding a complication that has occurred in the past and a score regarding the prognosis calculated for the complication in association with each other; and output information on the complication identified by the identification unit and information on the similar case retrieved from the storage unit.

An information processing method according to another aspect comprising: acquiring medical information collected when catheter treatment is performed on a patient; calculating a score related to a prognosis for each of a plurality of complications that may occur according to the catheter treatment on the basis of the acquired medical information; identifying one or more complications at risk among the plurality of complications on the basis of the calculated score; retrieving a similar case having a score similar to the calculated score from a storage unit configured to store information regarding a complication that has occurred in the past and a score related to the prognosis calculated for the complication in association with each other; and outputting information on the identified complication and information on the similar case retrieved from the storage unit.

A non-transitory computer-readable medium storing a program according to a further aspect, which when executed by a computer, performs processing comprising: acquiring medical information collected when catheter treatment is performed on a patient; calculating a score related to a prognosis for each of a plurality of complications that may occur according to the catheter treatment on the basis of the acquired medical information; identifying one or more complications at risk among the plurality of complications on the basis of the calculated score; retrieving a similar case having a score similar to the calculated score from a storage unit configured to store information regarding a complication that has occurred in the past and a score related to the prognosis calculated for the complication in association with each other; and outputting information on the identified complication and information on the similar case retrieved from the storage unit.

In one aspect, the risk regarding complications can be evaluated on the basis of various medical information collected at the time of catheter treatment.

### BRIEF DESCRIPTION OF THE DRAWINGS

FIG. 1 is a schematic diagram illustrating a configuration of a treatment support system.

FIG. 2 is a block diagram illustrating a configuration example of a treatment support device.

FIG. 3 is a conceptual diagram illustrating an example of a complication database.

FIG. 4 is an explanatory diagram for explaining a method of calculating a first score.

FIG. 5 is an explanatory diagram for explaining a method of calculating a second score.

FIG. 6 is a schematic diagram illustrating a configuration example of a learning model.

FIG. 7 is a schematic diagram illustrating a display example of complication information.

FIG. 8 is a schematic diagram illustrating a display example of complication information.

FIG. 9 is a flowchart illustrating a procedure of processing executed by the treatment support device.

FIG. 10 is a schematic diagram illustrating a display example of complication information in Second Embodiment.

FIG. 11 is a schematic diagram illustrating an example of a parameter change screen.

### DETAILED DESCRIPTION

Set forth below with reference to the accompanying drawings is a detailed description of embodiments of an information processing device, an information processing method, and a computer program.

### First Embodiment

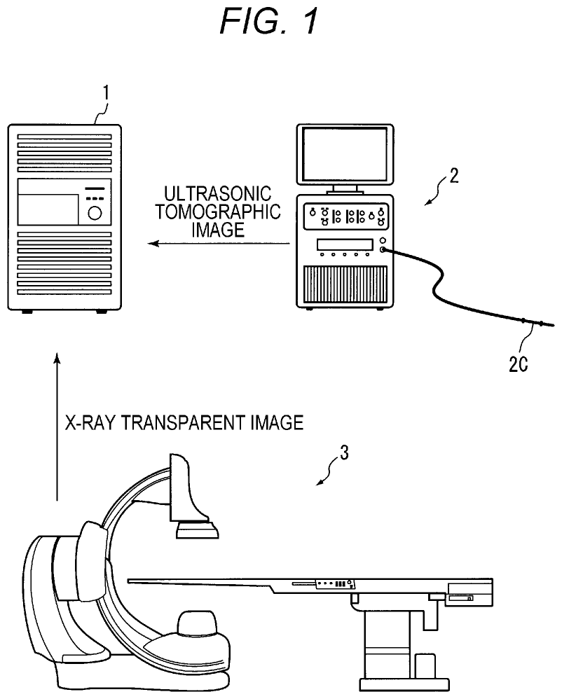

FIG. 1 is a schematic diagram illustrating a configuration of a treatment support system. In the embodiment, a description will be given of a treatment support system that identifies a complication having a high risk of poor prognosis on the basis of medical information collected when catheter treatment is performed, and provides a medical worker with information on the identified complication and information on a similar case. The treatment support system includes a treatment support device (or treatment support apparatus) 1, an intravascular image diagnostic device 2, a transparent image photographing device 3, and the like.

The treatment support device 1 is an information processing device such as a server computer or a personal computer. The treatment support device 1 can be installed, for example, in a medical facility that performs catheter treatment. Alternatively, the treatment support device 1 may be provided outside a medical facility and transmit and receive various types of information by communication. The treatment support device 1 acquires medical information collected when catheter treatment is performed on a patient, and identifies a complication at risk of poor prognosis on the basis of the acquired medical information. Here, the medical information includes attribute information of the patient, measurement information measured for the patient, and a medical image captured for the patient. The attribute information can be, for example, information such as age, sex, risk factor, and past medical history of the patient. The measurement information can be, for example, information such as blood test, lesion site, and stenosis rate. The medical image can be, for example, an image such as an ultrasonic tomographic image, an optical coherence tomographic image, an angiographic image, a computed tomography (CT) image, or a magnetic resonance imaging (MRI) image. The medical information may be collected in part prior to the catheter treatment and in other part during execution of the catheter treatment.

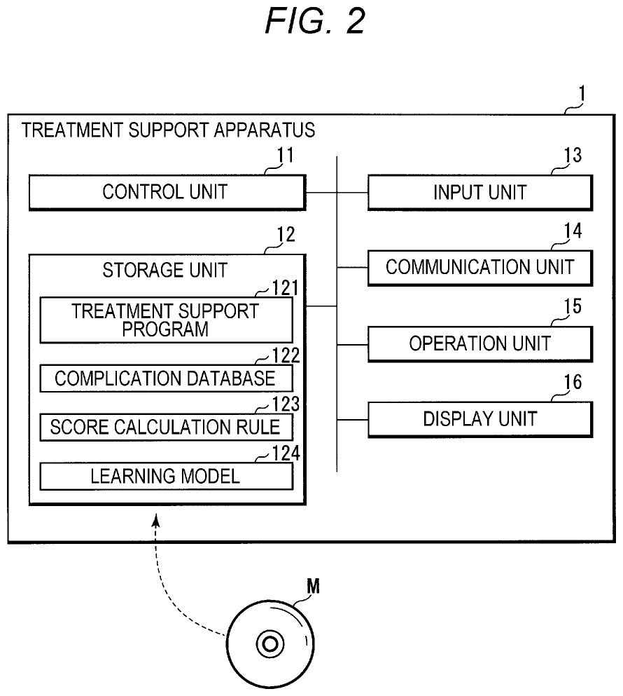

The treatment support device 1 provides a medical worker with information on complications at risk of poor prognosis identified on the basis of medical information. The treatment support device 1 can display, for example, information on complications at risk of poor prognosis on the display unit 16 (see FIG. 2). In addition, the treatment support device 1 may transmit information on complications at risk of poor prognosis to a terminal device such as a personal computer or a tablet terminal used by a medical worker.

In addition, the treatment support device 1 can search a database (the complication database 122 in FIG. 2) for a similar case similar to a complication having a risk of poor prognosis, and provides a medical worker with information on the searched similar case. The treatment support device 1 displays, for example, information on similar cases on the display unit 16. In addition, the treatment support device 1 may transmit information on similar cases to a terminal device such as a personal computer or a tablet terminal used by a medical worker.

At the time of treatment or diagnosis, the treatment support device 1 provides a medical worker with information on a complication having a risk of poor prognosis or information on a similar case having occurred in the past, thereby performing treatment support regarding catheter treatment.

The treatment support system includes an intravascular image diagnostic device 2 and a transparent image photographing device 3 as an example of a device that generates a medical image.

The intravascular image diagnostic device 2 is a device for obtaining an intravascular tomographic image of a patient, and includes, for example, an IVUS (Intravascular Ultrasound) device that performs an ultrasonic inspection using a catheter 2C. The catheter 2C is a medical instrument inserted into a blood vessel of a patient, and includes an imaging core that transmits ultrasounds and receives reflected waves from the blood vessel. The intravascular image diagnostic device 2 generates an ultrasound tomographic image (also referred to as a lateral tomographic image or an IVUS image) on the basis of the signal of the reflected wave received by the catheter 2C, and outputs the generated ultrasound tomographic image to the treatment support device 1. The treatment support device 1 causes the display unit 16 to display the ultrasound tomographic image input from the intravascular image diagnostic device 2 as necessary.

In the present embodiment, the intravascular image diagnostic device 2 generates an ultrasound diagnostic layer image, but may generate an optical coherence tomography image by an optical method such as optical coherence tomography (OCT) or optical frequency domain imaging (OFDI).

The transparent image photographing device 3 is a device unit for capturing a transparent image of the inside of the patient body, and can include, for example, an angiography device that performs an angiography examination. The transparent image photographing device 3 can include an X-ray source, an imaging plate, and the like. The transparent image photographing device 3 generates an X-ray transparent image (also referred to as an angiographic image) by detecting the X-ray emitted from the X-ray source and transmitted through the irradiation site with the imaging plate. In the catheter treatment, the radiopaque marker may be attached to the distal end of the catheter 2C, and the position of the radiopaque marker may be used for alignment with the tomographic image generated by the intravascular image diagnostic device 2.

The treatment support system is not limited to the above-described configuration, and may include a CT device that generates a CT image, an MRI device that generates an MRI image, and various measuring instruments that measure the condition of a patient during surgery.

FIG. 2 is a block diagram illustrating a configuration example of the treatment support device (or treatment support apparatus) 1. The treatment support device 1 includes a control unit 11, a storage unit 12, an input unit 13, a communication unit 14, an operation unit 15, a display unit 16, and the like.

The control unit 11 includes, for example, a central processing unit (CPU), a read only memory (ROM), a random access memory (RAM), and the like. The ROM included in the control unit 11 stores a control program and the like for controlling the operation of each unit of hardware included in the treatment support device 1. The CPU in the control unit 11 executes a control program stored in the ROM and various computer programs stored in the storage unit 12 to be described later, and controls the operation of each unit of the hardware, thereby causing the entire device to function as the information processing device of the present application. Data generated during the execution of the arithmetic operation can be temporarily stored in the RAM included in the control unit 11.

The control unit 11 can include a CPU, a ROM, and a RAM, but may be one or a plurality of arithmetic circuits including a graphics processing unit (GPU), a field programmable gate array (FPGA), a digital signal processor (DSP), a quantum processor, a volatile or nonvolatile memory, and the like. Furthermore, the control unit 11 may have functions such as a clock that outputs date and time information, a timer that measures an elapsed time from when a start instruction is given to when an end instruction is given, and a counter that counts the number.

The storage unit 12 includes a storage device such as a hard disk drive (HDD) or a solid state drive (SSD). The storage unit 12 stores various computer programs executed by the control unit 11 and various data used by the control unit 11.

The computer program stored in the storage unit 12 includes a treatment support program 121. The treatment support program 121 is a computer program for causing the treatment support device 1 to execute processing of identifying a complication at risk of poor prognosis on the basis of the acquired medical information and outputting information on the identified complication and information on a similar case obtained from the complication database 122.

The computer program stored in the storage unit 12 is provided by a non-transitory recording medium M in which the computer program is readably recorded. The recording medium M can be, for example, a portable memory such as a CD-ROM, a USB memory, a secure digital (SD) card, and a compact flash®. The control unit 11 reads a computer program recorded on the recording medium M using a reading device, and stores the read computer program in the storage unit 12. Alternatively, the computer program stored in the storage unit 12 may be provided by communication. In this case, the control unit 11 may acquire the computer program through the communication unit 14 and store the acquired computer program in the storage unit 12.

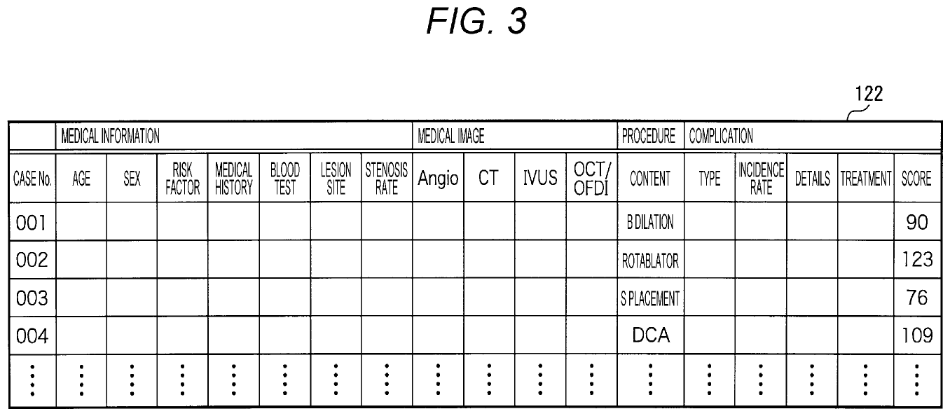

In addition, the storage unit 12 includes a complication database 122. FIG. 3 is a diagram illustrating an example of the complication database 122. The complication database 122 stores medical information, medical images, procedure information, and complication information in association with complications that have occurred due to catheter treatment.

The medical information can include attribute information of the patient including age, sex, risk factor, medical history, and the like, and measurement information measured for the patient including a blood test, a lesion site, a stenosis rate, and the like. The medical image can include an angiographic image, a CT image, an IVUS image (ultrasonic tomographic image), an OCT/OFDI image (optical coherence tomographic image), and the like. In the complication database 122, information indicating a link specifying an image file related to these images and a storage location is registered.

The procedure information is information on a procedure performed at the time of catheter treatment. Examples of the procedure used for the catheter treatment include balloon dilation, stent placement, directional coronary artery resection, and cutting of a calcified lesion using a rotablator.

The complication information can include information such as the type, incidence rate, details, treatment, score, and the like of the complication. The complications that may occur according to catheter treatment include dissection, side branch occlusion, No Reflow, Slow Flow, etc. The incidence rate indicates the rate at which complications occurred relative to catheter treatment. For example, when the total number of catheter treatments is 1000 and the number of occurrences of dissection is three, the incidence rate of dissection is 0.3%. The details are detailed information of the complication, and the treatment is information of treatment performed for the complication. The score is obtained by quantifying the quality of the prognosis of the catheter treatment, and can be calculated using a score calculation rule 123 and a learning model 124 to be described later.

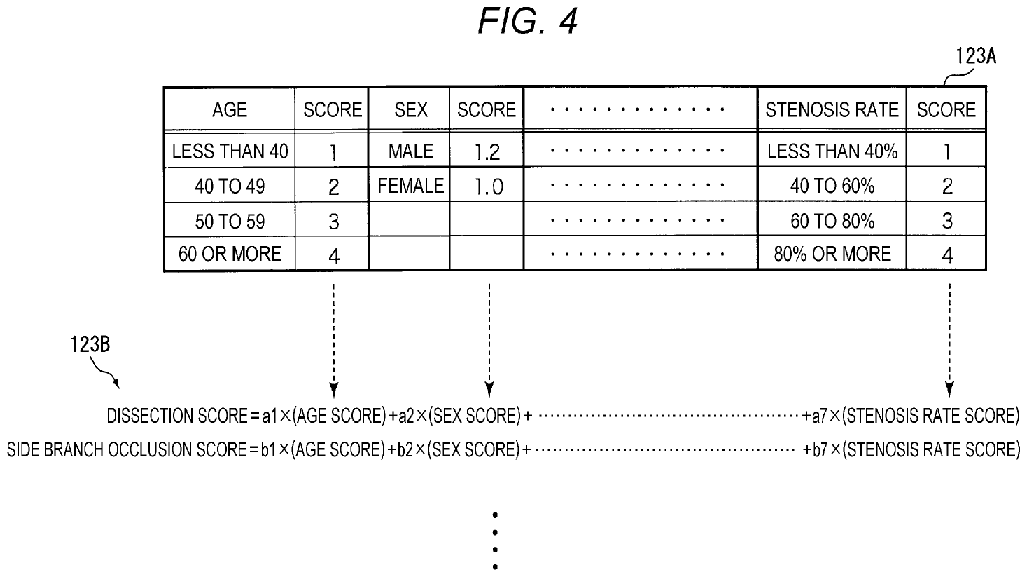

The storage unit 12 stores the score calculation rule 123. The score calculation rule 123 can include, for example, a numerical table 123A for quantifying the above-described medical information and a score calculation formula 123B for calculating a score (first score) related to a prognosis (for example, a good or bad prognosis) of the catheter treatment on the basis of the quantified medical information (see FIG. 4). A method of calculating the first score based on the medical information will be described in detail later.

The storage unit 12 includes a learning model 124. The learning model 124 is a learning model of machine learning configured to output a score (second score) related to a prognosis (for example, good or bad prognosis) for a complication that may occur according to catheter treatment in a case where a medical image is input. The learning model 124 can be constructed by, for example, convolutional neural networks (CNN). The storage unit 12 stores, as information defining the learning model 124, information on layers constituting the neural network, information on nodes constituting each layer, and information on weights and biases set between nodes. In the present embodiment, it is assumed that learning is performed in advance so that the learning model 124 outputs a score (second score) related to the prognosis (e.g., good or bad prognosis) according to the input of the medical images by using, as training data, a data set including a plurality of sets of medical images captured at the time of performing catheter treatment and a doctor's diagnosis result indicating a prognosis (good or bad of the prognosis).

The learning model 124 is not limited to the learning model constructed by the CNN, and may be a learning model based on a region-based CNN (R-CNN), a You Only Look Once (YOLO), a single shot detector (SSD), a Generative Adversarial Network (GAN), a support vector machine (SVM), a decision tree, or the like.

The input unit 13 includes an interface for connecting the intravascular image diagnostic device 2 and the transparent image photographing device 3, and acquires an ultrasound tomographic image generated by the intravascular image diagnostic device 2 and an angiographic image generated by the transparent image photographing device 3. In the present embodiment, a medical image is acquired from the intravascular image diagnostic device 2 or the transparent image photographing device 3 connected to the input unit 13, but a medical image captured by another computer may be acquired by communication. Furthermore, a measuring instrument that measures the condition of the patient may be connected to the input unit 13, and measurement information by the measuring instrument may be input.

The communication unit 14 includes a communication interface that transmits and receives various data. The communication interface included in the communication unit 14 can be, for example, a communication interface conforming to a communication standard of a local area network (LAN) used in WiFi® or Ethernet®. When data to be transmitted is input from the control unit 11, the communication unit 14 transmits the data to be transmitted to a designated destination. Furthermore, when receiving data transmitted from an external device, the communication unit 14 outputs the received data to the control unit 11.

The operation unit 15 can include an operation device such as a keyboard, a mouse, and a touch panel, and receives various operations and settings by a medical worker or the like. The control unit 11 performs appropriate control on the basis of various operation information given from the operation unit 15, and stores setting information in the storage unit 12 as necessary.

The display unit 16 can include a display device such as a liquid crystal monitor or an organic electro-luminescence (EL), and displays information to be notified to a medical worker or the like in response to an instruction from the control unit 11.

Although the configuration example of the treatment support device 1 has been described with reference to FIG. 2, the configuration of the treatment support device 1 is not limited to the above. For example, the complication database 122 and the learning model 124 may be stored in an external storage device accessible from the treatment support device 1. In this case, the treatment support device 1 may access the external storage device via the communication unit 14 to acquire necessary information from the complication database 122 or execute calculation using the learning model 124. In addition, the operation unit 15 and the display unit 16 are not essential in the treatment support device 1, and may be configured to receive an operation from an external computer communicably connected to the treatment support device 1 and display various types of information on an external monitor. The external computer may be the intravascular image diagnostic device 2, and the external monitor may be a monitor included in the intravascular image diagnostic device 2. Furthermore, the treatment support device 1 does not need to be a single computer, and may be a multi-computer including a plurality of computers. Furthermore, the treatment support device 1 may be a virtual machine virtually constructed by software.

Hereinafter, processing contents in the treatment support device 1 will be described.

The treatment support device 1 identifies one or a plurality of complications at risk of poor prognosis among a plurality of complications that may occur according to catheter treatment, and provides information on the identified complications to a medical worker. At this time, the treatment support device 1 calculates a score related to a prognosis (good or bad of prognosis) for each of a plurality of possible complications on the basis of medical information collected at the time of catheter treatment. As described below, the score related to the prognosis (good or bad of prognosis) is calculated from the first score based on the attribute information and the measurement information of the patient and the second score based on the medical image.

FIG. 4 is an explanatory diagram illustrating a method of calculating a first score. The control unit 11 of the treatment support device 1 quantifies the attribute information of the patient and the measurement information measured for the patient among the medical information collected for the patient. The control unit 11 can quantify the attribute information and the measurement information of the patient by referring to the numerical table 123A as illustrated in FIG. 4.

For example, the control unit 11 refers to the numerical table 123A, and can quantify the age of the patient, for example, as “1” in a case where the patient's age is less than 40 years old, “2” in a case of the patient in 40's, “3” in a case of the patient in 50's, and “4” in a case of the patient in 60's or older. The same applies to other attribute information such as sex, risk factor, and medical history of the patient.

In addition, the control unit 11 refers to the numerical table 123A, and can quantify the stenosis rate, for example, as “1” when the measured stenosis rate is less than 40%, “2” when the measured stenosis rate is 40% or more and less than 60%, “3” when the measured stenosis rate is 60% or more and less than 80%, and “4” when the measured stenosis rate is 80% or more. The same applies to other measurement information such as a blood test and a lesion site.

In the present embodiment, the attribute information and the measurement information of the patient are quantified using the numerical table 123A. However, instead of the quantification using the numerical table 123A, the quantification may be performed using a preset calculation formula or function.

The control unit 11 calculates the first score for each complication by substituting each piece of quantified medical information into the score calculation formula 123B for each complication. The score calculation formula 123B is represented by, for example, a weighted sum of numerical values obtained by quantifying each piece of medical information. Not limited to the weighted sum, other calculation formulas may be used.

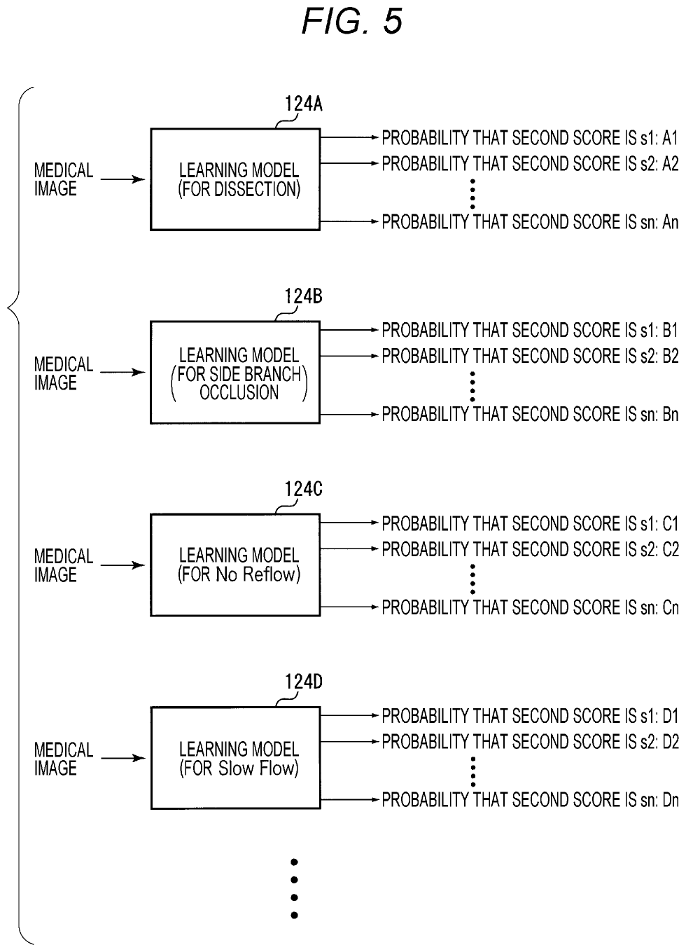

FIG. 5 is an explanatory diagram illustrating a method of calculating a second score. The control unit 11 of the treatment support device 1 calculates the second score based on the medical image using the learning model 124. In the present embodiment, the learning model 124 is prepared for each complication. In the example illustrated in FIG. 5, learning models 124A to 124D represent learning models for dissection, side branch occlusion, No Reflow, and Slow Flow, respectively. The learning models 124A to 124D are configured to output information of the second score regarding dissection, side branch occlusion, No Reflow, and Slow Flow, respectively, with respect to the input of the medical image.

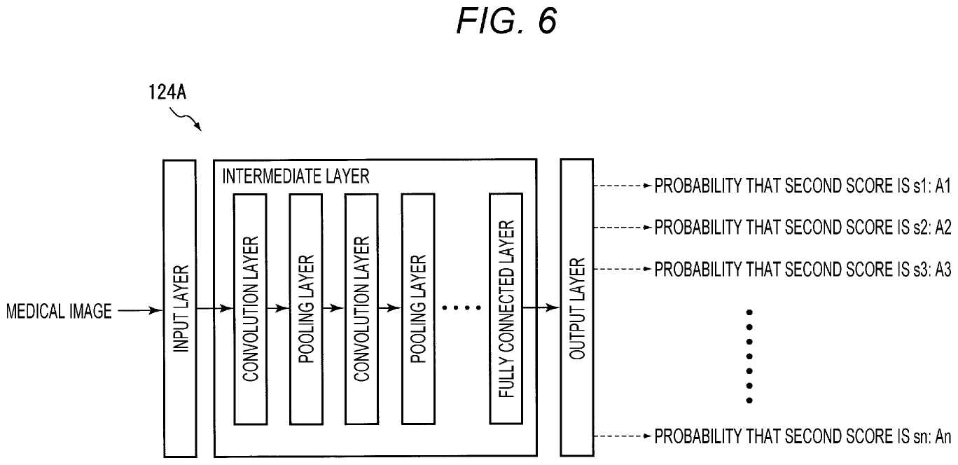

FIG. 6 is a schematic diagram illustrating a configuration example of the learning model 124A. The learning model 124A includes an input layer, an intermediate layer (hidden layer), and an output layer. Image data of a medical image is input to the input layer. The image data of the medical image input to the input layer is sent to the intermediate layer.

The intermediate layer includes a convolution layer, a pooling layer, and a fully connected layer. A plurality of convolution layers and a plurality of pooling layers may be alternately provided. The convolution layer and the pooling layer extract features of the medical image input from the input layer by calculation using nodes of the respective layers. The fully connected layer connects the data in which the feature portion is extracted by the convolution layer and the pooling layer to one node, and outputs the feature variable converted by the activation function. The feature variable is output to the output layer through the fully connected layer.

The output layer includes one or more nodes. The output layer converts the feature variable input from the fully connected layer of the intermediate layer into a probability using a softmax function, and outputs the probability corresponding to each score from each node. For example, the first node outputs the probability (=A1) that the score is s1, the second node outputs the probability (=A2) that the score is s2, . . . , and the n-th node outputs the probability (=An) that the score is sn. The control unit 11 refers to the probability of each score output from the output layer of the learning model 124, and determines a score having the highest probability as a score (second score) based on the medical image.

In the example of FIG. 6, the learning model 124A that outputs the information of the second score regarding the dissection has been described, but the same applies to the learning models 124B to 124D regarding side branch occlusion, No Reflow, and Slow Flow. The learning model 124 provided in the treatment support device 1 is not limited to the above-described four learning models 124A to 124D, and is prepared for each of a plurality of complications that may occur according to catheter treatment. In addition, a learning model may be prepared for each medical image such as an ultrasonic tomographic image, an optical coherence tomographic image, an angiographic image, a CT image, or an MRI image, and the learning model to be used may be switched according to the acquired medical image.

In a case where the first score for each complication calculated from the medical information (attribute information and measurement information) of the patient and the second score for each complication calculated from the medical image are obtained, the control unit 11 calculates a final score (total score) for each complication from the obtained first score and second score. The total score may be a sum of the first score and the second score, or may be a weighted sum.

The total score represents the quality of prognosis for each complication. In the present embodiment, for example, the higher the total score, the worse the prognosis. The control unit 11 identifies one or a plurality of complications at risk of poor prognosis on the basis of the total score. For example, the control unit 11 identifies a complication at risk of poor prognosis by selecting a predetermined number of complications in order from the highest total score. In addition, the control unit 11 may compare the total score of each complication with a preset threshold value and identify a complication having a total score equal to or more than the threshold value as a complication at risk of poor prognosis.

Since the treatment support device 1 includes the complication database 122 that stores information on complications having occurred in the past and the score (total score) calculated for the complications in association with each other, it is possible to retrieve a similar case from the complication database 122 on the basis of the total score calculated as described above. That is, the control unit 11 may retrieve a complication having a score similar to the total score calculated on the basis of the medical information as a similar case. The similarity/dissimilarity of the scores may be determined on the basis of whether or not a difference (or ratio) between the calculated total score and the score registered in the complication database 122 falls within a predetermined range.

The control unit 11 outputs the information on the complication at risk of poor prognosis identified from the medical information and the information on the similar case retrieved from the complication database 122, and displays the information on the display unit 16.

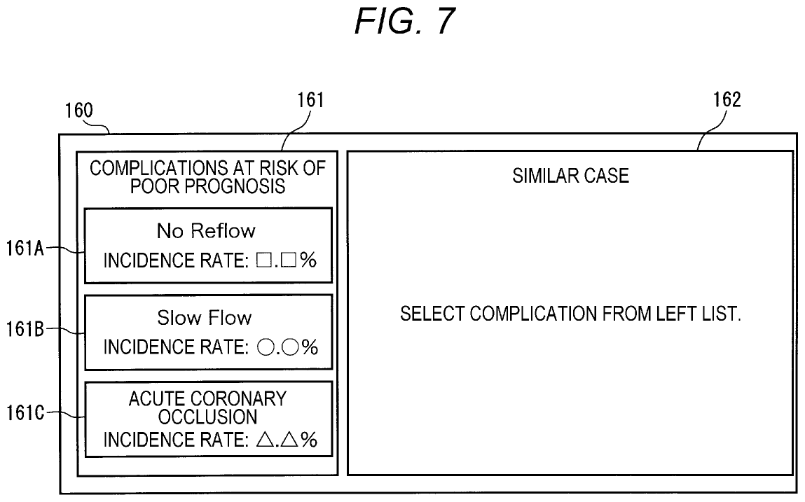

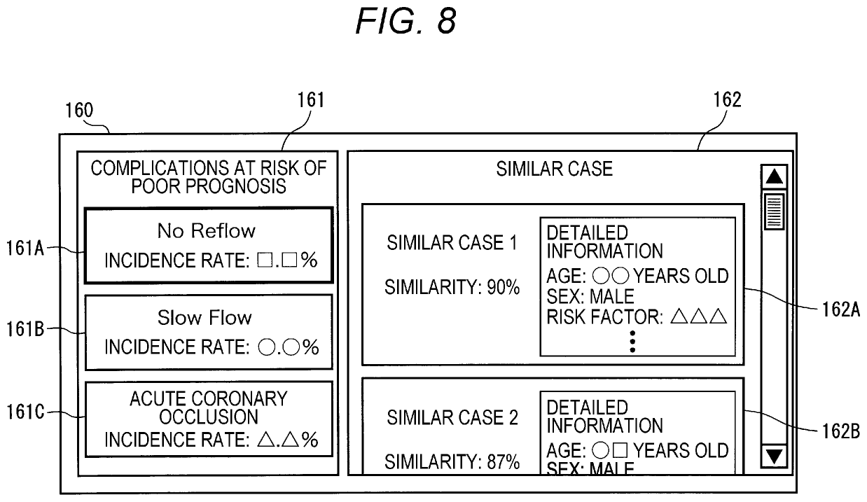

FIGS. 7 and 8 are schematic diagrams illustrating display examples of complication information. FIGS. 7 and 8 illustrate an example in which a display screen 160 of the complication information is displayed on the display unit 16 of the treatment support device 1. The display screen 160 can include a complication list 161 that displays complications at risk of poor prognosis in the form of a list and a similar case list 162 that displays similar cases in the form of a list.

In the example of FIG. 7, information on complications at risk of poor prognosis is displayed in the complication list 161, and information on similar cases is not displayed in the similar case list 162. In the complication list 161, it can be preferable to display a predetermined number (three in the example of FIG. 7) of pieces of information on complications in descending order of risk of poor prognosis. The quality of prognosis of each complication can be determined by the total score described above. The complication list 161 can include, for example, a first panel 161A that displays information on a complication having the highest risk of poor prognosis (No Reflow in the example of FIG. 7), a second panel 161B that displays information on a complication having the second highest risk of poor prognosis (Slow Flow in the example of FIG. 7), and a third panel 161C that displays information on a complication having the third highest risk of poor prognosis (acute coronary occlusion in the example of FIG. 7). In each of the panels 161A to 161C, the name of the complication and information on the incidence rate in the medical institution are displayed as the information on the complication.

FIG. 7 illustrates an example in which the name of the complication and the information of the incidence rate are displayed as the information on the complication at risk of poor prognosis, but other information such as a total score may be displayed together. In addition, the number of complications to be displayed is not limited to three, and information on one or more complications may be displayed.

Each of the panels 161A to 161C arranged in the complication list 161 is configured to be selectable using the operation unit 15. If any one of the panels 161A to 161C is selected, similar cases are displayed in a list form in the similar case list 162 for the complications displayed in the selected panel 161A (or 161B, 161C).

The example of FIG. 8 illustrates a state in which the panel 161A is selected on the display screen 160 illustrated in FIG. 7. Since the complication displayed in the panel 161A is No Reflow, information on similar cases of No Reflow is displayed in the similar case list 162. In the similar case list 162, similar cases are displayed in descending order of similarity to the selected complication. The similarity can be calculated by, for example, |x−y|/y×100 using the total score (x) of the selected complication and the total score (y) of the similar case calculated for each similar case.

In the example of FIG. 8, two panels 162A and 162B are displayed in the similar case list 162. A similar case (similar case 1) having the highest similarity is displayed in the first panel 162A, and a similar case (similar case 2) having the second highest similarity is displayed in the second panel 162B. Other similar cases can be displayed by scrolling the scroll bar at the right end. In each of the panels 162A and 162B, the name of the similar case, the similarity, and the detailed information of the similar case are displayed as the information of the similar case. The detailed information can include attribute information of a patient, measurement information, and information on the performed procedure and treatment. In addition, a link for displaying a medical image may be provided in each of the panels 162A and 162B, and the medical image may be displayed when the link is operated.

Hereinafter, a procedure of processing executed by the treatment support device 1 will be described.

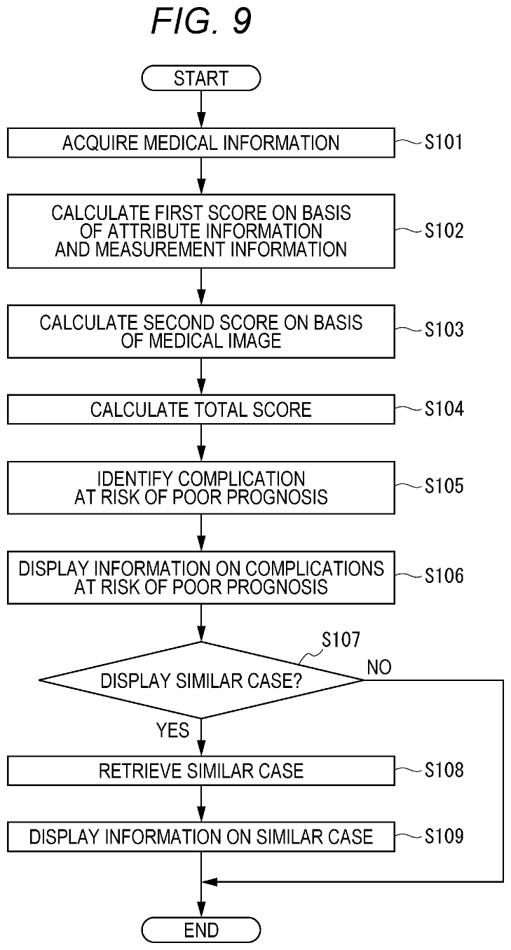

FIG. 9 is a flowchart illustrating a procedure of processing executed by the treatment support device 1. The control unit 11 of the treatment support device 1 executes the following processing by reading and executing the treatment support program 121 from the storage unit 12.

The control unit 11 acquires medical information collected when catheter treatment is performed on a patient (S101). The medical information can include attribute information of the patient, measurement information measured for the patient, and a medical image captured for the patient. The control unit 11 can receive attribute information such as the age and sex of the patient through the operation unit 15. Furthermore, the control unit 11 can acquire measurement information measured for the patient and a medical image captured for the patient through the input unit 13. The control unit 11 may acquire medical information at an appropriate timing before or after the start of catheter treatment. It is not necessary to acquire all the medical information at the same timing, and necessary medical information may be sequentially acquired at an appropriate timing.

Next, the control unit 11 calculates the first score using the attribute information and the measurement information included in the acquired medical information (S102). That is, the control unit 11 quantifies the attribute information and the measurement information of the patient with reference to the numerical table 123A and substitutes the quantified information into the score calculation formula 123B defined for each complication to calculate the first score for each complication.

Next, the control unit 11 calculates the second score using the medical image included in the acquired medical information (S103). That is, the control unit 11 inputs the medical image acquired in S101 to the learning model 124 (the learning models 124A to 124D in the example of FIG. 5) for each complication, and determines the second score for each complication on the basis of the information output from the learning model 124.

Next, the control unit 11 calculates a score (total score) related to the prognosis (good or bad of prognosis) for each complication by using the first score calculated in S102 and the second score calculated in S103 (S104). The total score may be a sum of the first score and the second score, or may be a weighted sum.

Next, the control unit 11 identifies one or a plurality of complications at risk of poor prognosis among a plurality of complications that may occur according to the catheter treatment on the basis of the calculated total score (S105). For example, the control unit 11 identifies a complication at risk of poor prognosis by selecting a predetermined number (for example, three) of complications in order from the highest total score. Alternatively, the control unit 11 may identify a complication at risk of poor prognosis, for example, by selecting a complication having a total score equal to or higher than a threshold value.

Next, the control unit 11 causes the display unit 16 to display information on complications at risk of poor prognosis identified in S105 (S106). At this time, the control unit 11 outputs display data for displaying the names of the identified complications in order of scores to the display unit 16, and causes the display unit 16 to display the complication list 161. In addition, the control unit 11 may read the incidence rate of the complication from the complication database 122, output display data for displaying the incidence rate of the complication to the display unit 16 together with the name of the complication, and display the name and the incidence rate of the complication in the complication list 161.

Next, the control unit 11 determines whether or not to display a similar case (S107). In a case where a specific complication is selected in the complication list 161 displayed on the display unit 16, the control unit 11 determines to display a similar case for the complication. When determining not to display the similar case (S107: NO), the control unit 11 ends the processing according to this flowchart.

When determining to display the similar case (S107: YES), the control unit 11 searches the complication database 122 for a complication (similar case) having a total score similar to the selected complication (S108). Since the complication database 122 stores information on complications having occurred in the past and the total score calculated for the complications in association with each other, the control unit 11 searches the complication database 122 for a complication having a score similar to the selected complication as a similar case. The similarity/dissimilarity of the scores may be determined depending on whether or not a difference (or ratio) between the calculated total score and the score registered in the complication database 122 falls within a predetermined range.

In the present embodiment, the similar case is searched from the complication database 122 when it is determined that the similar case is to be displayed. However, the similar case may be searched at the time when a complication at risk of poor prognosis is identified in S105.

The control unit 11 causes the display unit 16 to display the information on the similar case searched in S108 (S109). At this time, the control unit 11 outputs display data for displaying the searched similar cases in order of similarity to the display unit 16, and causes the display unit 16 to display information on similar cases in the similar case list 162 of the display unit 16. In addition, the control unit 11 may read the similarity and the information on the catheter treatment that has caused the complication from the complication database 122, and cause the display unit 16 to display the name of the similar case and the information on the catheter treatment that has caused the complication in the similar case list 162.

The control unit 11 may perform the above processing before the catheter treatment and provide the medical worker with information on complications and similar cases as support information when a treatment strategy is formulated. Furthermore, the control unit 11 may perform the above-described processing during execution of the catheter treatment and provide the medical worker with information on complications and similar cases as support information at the time of surgery. Furthermore, the control unit 11 may perform the above-described processing after execution of the catheter treatment, and provide the medical worker with information on complications and similar cases as support information when performing treatment evaluation.

As described above, the treatment support device 1 according to the present embodiment can present information related to a risk of poor prognosis regarding a complication that may occur regarding catheter treatment to a medical worker or the like. As a result, it is possible to improve the treatment accuracy, avoid erroneous determination, and shorten the operation time, which is expected to contribute to improvement of the quality of catheter treatment.

In the present embodiment, the first score is calculated on a rule basis, and the second score is calculated using the learning model 124. However, the score related to the prognosis (good or bad of prognosis) may be calculated using only the machine learning model. That is, in a case where the attribute information, the measurement information, and the medical image of the patient included as the medical information are input, the control unit 11 may calculate the score by using a learning model configured to output information regarding the score related to the prognosis (good or bad of prognosis).

Furthermore, in the present embodiment, the learning models 124A to 124D for each complication are prepared and the second score for each complication is calculated. However, in a case where a medical image is input, the second score of each complication may be calculated using one learning model configured to output information regarding the second score of each complication.

Furthermore, in the present embodiment, the score is calculated using the attribute information, the measurement information, and the medical image of the patient. However, a set value or a measured value (For example, balloon pressure) in an device such as the intravascular image diagnostic device 2 or the transparent image photographing device 3 may be acquired, and the score may be calculated in consideration of the acquired set value or measured value.

### Second Embodiment

In a second embodiment, a configuration in which filtering is performed according to a parameter included in medical information and filtered information is displayed will be described.

Since the overall configuration of the treatment support system and the internal configuration of the treatment support device 1 are similar to those of the first embodiment, the description of the overall configuration of the treatment support system and the internal configuration of the treatment support device 1 will be omitted.

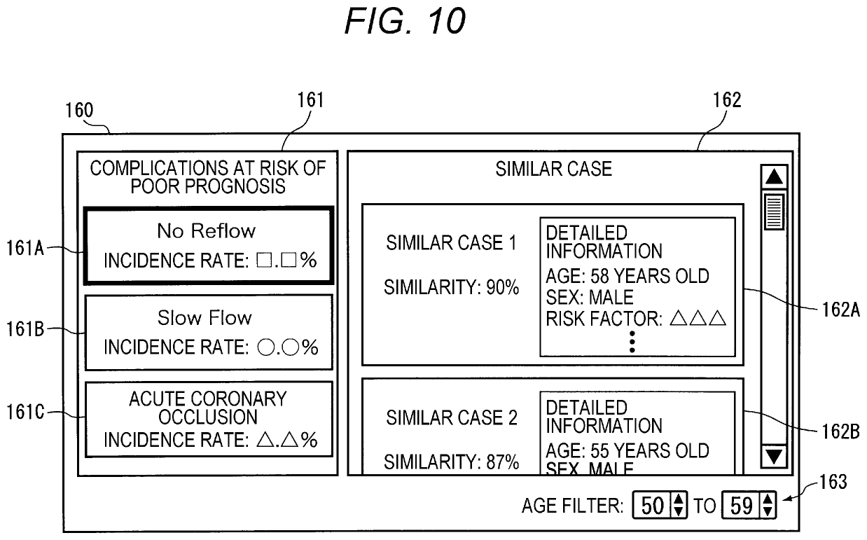

FIG. 10 is a schematic diagram illustrating a display example of complication information in the second embodiment. FIG. 10 illustrates an example in which the display screen 160 of the complication information is displayed on the display unit 16 of the treatment support device 1. The display screen 160 can include, for example, an age filter 163 in addition to the complication list 161 and the similar case list 162. In a case where the lower limit value and the upper limit value of age are set in the age filter 163, the control unit 11 searches the complication database 122 for a complication (similar case) that has occurred in a patient of a corresponding age, and displays similar case information obtained as a search result in the similar case list 162. In the example of FIG. 10, since the lower limit value is set to 50 years old and the upper limit value is set to 59 years old, the control unit 11 searches the complication database 122 for complications that have occurred in patients of 50 years old or more and 59 years old or less, and displays similar case information obtained as a search result in the similar case list 162.

In the present embodiment, the configuration in which the age filter 163 is provided on the display screen 160 has been described, but filtering may be performed by other medical information including sex, risk factor, medical history, blood test, lesion site, and stenosis rate, and medical images including an angiographic image, a CT image, an IVUS image, and an OCT/OFDI image.

As described above, in the present embodiment, since filtering can be performed according to the parameters included in the medical information, it is possible to provide the medical worker with information on similar cases having closer conditions.

### Third Embodiment

In a third embodiment, a configuration for displaying how much a score changes according to a change in a parameter included in medical information will be described.

Since the overall configuration of the treatment support system and the internal configuration of the treatment support device 1 are similar to those of the first embodiment, the description of the overall configuration of the treatment support system and the internal configuration of the treatment support device 1 will be omitted.

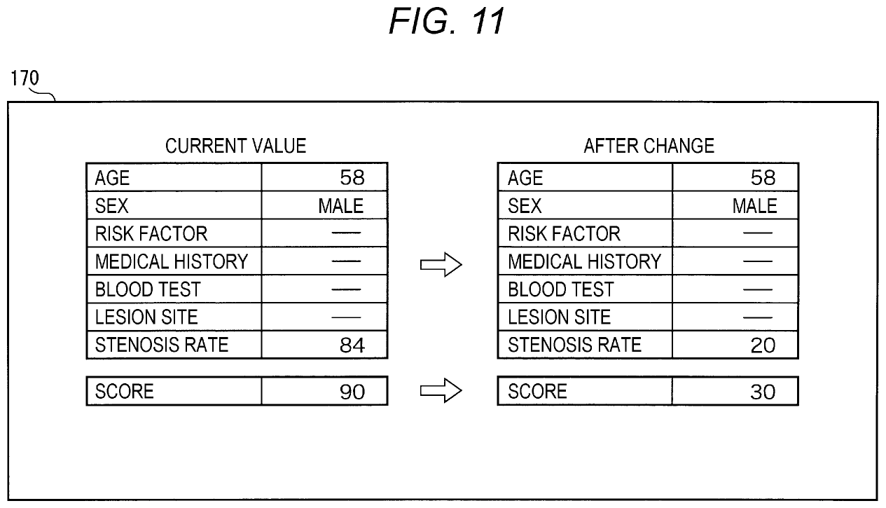

FIG. 11 is a schematic diagram illustrating an example of a parameter change screen. FIG. 11 illustrates an example in which a parameter change screen 170 is displayed on the display unit 16 of the treatment support device 1. The treatment support device 1 can receive a change in attribute information and measurement information of a patient through the change screen 170 displayed on the display unit 16. In a case where one of the parameters included in the attribute information and the measurement information is changed, the control unit 11 of the treatment support device 1 recalculates the score and displays the recalculated score on the change screen 170. The example of FIG. 11 shows that the score decreased from 90 to 30 as a result of changing the stenosis rate from 84% to 20%. Note that the score to be calculated may be a first score (or a second score) or a total score.

Further, not only the change of the attribute information and the measurement information of the patient but also the change of the medical image may be received. Furthermore, changes in various setting values and measured values (for example, the pressure of the balloon and the like) in devices such as the intravascular image diagnostic device 2 and the transparent image photographing device 3 may be received, and the score may be recalculated on the basis of the changed parameters.

As described above, in the present embodiment, it is possible to present how the score changes in a case where the parameter included in the medical information is changed. Therefore, the medical worker can appropriately formulate a treatment strategy by referring to the information provided through the change screen 170.

The detailed description above describes embodiments of an information processing device, an information processing method, and a computer program. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. An information processing device comprising:
a processor configured to:
acquire medical information collected when catheter treatment is performed on a patient;
calculate a score related to a prognosis for each of a plurality of complications that may occur according to the catheter treatment on the basis of the acquired medical information;
identify one or more complications at risk among the plurality of complications on the basis of the calculated score;
retrieve a similar case having a score similar to the score calculated from a storage unit configured to store information regarding a complication that has occurred in the past and a score related to the prognosis calculated for the complication in association with each other; and
output information on the complication identified by the identification unit and information on the similar case retrieved from the storage unit.

2. The information processing device according to claim 1, wherein the processor is configured to:
output data for displaying names of the complications identified by the identification unit in order of scores.

3. The information processing device according to claim 1, wherein the processor is configured to:
output data for displaying an incidence rate of the complication in a medical institution in which the catheter treatment is performed together with the names of the complications identified by the identification unit.

4. The information processing device according to claim 1, wherein the processor is configured to:
receive a selection operation for the one or more complications identified by the identification unit; and
output data for displaying a list of information on similar cases of the selected complication.

5. The information processing device according to claim 1, wherein the processor is configured to:
output data for displaying the information on the similar cases retrieved from the storage unit in order of score similarities.

6. The information processing device according to claim 5, wherein the processor is configured to:
output, for the similar case, data for displaying the similarity of the score and information on catheter treatment that has caused a complication together.

7. The information processing device according to claim 1, wherein the processor is configured to:
output information on a complication identified by the identification unit and information on a similar case retrieved from the storage unit during execution of the catheter treatment.

8. The information processing device according to claim 1, wherein the medical information includes attribute information of the patient, measurement information measured for the patient, and a medical image captured for the patient.

9. The information processing device according to claim 8, wherein the processor is configured to:
calculate a first score on a rule basis using the attribute information and the measurement information;
input the medical image to a machine learning model to calculate a second score; and
calculate a score related to the prognosis of each complication on the basis of the calculated first score and second score.

10. An information processing method comprising:
acquiring medical information collected when catheter treatment is performed on a patient;
calculating a score related to a prognosis for each of a plurality of complications that may occur according to the catheter treatment on the basis of the acquired medical information;
identifying one or more complications at risk among the plurality of complications on the basis of the calculated score;
retrieving a similar case having a score similar to the calculated score from a storage unit configured to store information regarding a complication that has occurred in the past and a score related to the prognosis calculated for the complication in association with each other; and
outputting information on the identified complication and information on the similar case retrieved from the storage unit.

11. The information processing method according to claim 10, further comprising:
outputting data for displaying names of the complications identified by the identification unit in order of scores.

12. The information processing method according to claim 10, further comprising:
outputting data for displaying an incidence rate of the complication in a medical institution in which the catheter treatment is performed together with the names of the complications identified by the identification unit.

13. The information processing method according to claim 10, further comprising:
receiving a selection operation for the one or more complications identified by the identification unit; and
outputting data for displaying a list of information on similar cases of the selected complication.

14. The information processing method according to claim 10, further comprising:
outputting data for displaying the information on the similar cases retrieved from the storage unit in order of score similarities.

15. The information processing method according to claim 14, further comprising:
outputting, for the similar case, data for displaying the similarity of the score and information on catheter treatment that has caused a complication together.

16. The information processing method according to claim 10, further comprising:
outputting information on a complication identified by the identification unit and information on a similar case retrieved from the storage unit during execution of the catheter treatment.

17. The information processing method according to claim 10, wherein the medical information includes attribute information of the patient, measurement information measured for the patient, and a medical image captured for the patient.

18. The information processing method according to claim 17, further comprising:
calculating a first score on a rule basis using the attribute information and the measurement information;
inputting the medical image to a machine learning model to calculate a second score; and
calculating a score related to the prognosis of each complication on the basis of the calculated first score and second score.

19. A non-transitory computer-readable medium storing a program, which when executed by a computer, performs processing comprising:
acquiring medical information collected when catheter treatment is performed on a patient;
calculating a score related to a prognosis for each of a plurality of complications that may occur according to the catheter treatment on the basis of the acquired medical information;
identifying one or more complications at risk among the plurality of complications on the basis of the calculated score;
retrieving a similar case having a score similar to the calculated score from a storage unit configured to store information regarding a complication that has occurred in the past and a score related to the prognosis calculated for the complication in association with each other; and
outputting information on the identified complication and information on the similar case retrieved from the storage unit.

20. The non-transitory computer-readable medium according to claim 19, further comprising:
outputting data for displaying names of the complications identified by the identification unit in order of scores.
